(19) United States
(12) Patent Application Publication
(10) Pub. No.: US 2018/0256474 A1
(43) Pub. Date: Sep. 13, 2018

(54) COMPOSITION FOR DYEING KERATIN FIBERS

(71) Applicant: L'OREAL, Paris (FR)

(72) Inventors: Shiho MIYAKE, Kawasaki-shi, Kanagawa (JP); Hidetoshi YAMADA, Kawasaki-shi, Kanagawa (JP); Hidehiko ASANUMA, Kawasaki-shi, Kanagawa (JP)

(21) Appl. No.: 15/779,819

(22) PCT Filed: Nov. 25, 2016
(86) PCT No.: PCT/JP2016/085831

§ 371 Date: May 29, 2018

(30) Foreign Application Priority Data
Dec. 4, 2015 (JP) 2015-237355

(51) Int. Cl. A61K 8/49; A61K 8/46; A61K 8/35; A61Q 5/06

(52) U.S. Cl. CPC A61K 8/4973; A61K 8/466; A61K 8/4926; A61K 2800/5922; A61Q 5/065; A61K 2800/432; A61K 8/355

(57) ABSTRACT

The present invention relates to a composition for dyeing keratin fibers, comprising (a) at least one direct dye; and (b) at least one oxygen-containing polycyclic compound which has at least one oxygen-containing polycyclic ring. The composition according to the present invention may further include (c) at least one specific sulfone compound. The composition according to the present invention is a one-part composition and can prevent or reduce skin staining by the direct dye on the skin such as the scalp, while providing the keratin fibers with good cosmetic effects such as good coloring properties.

### TECHNICAL FIELD

The present invention relates to a composition for dyeing keratin fibers, in particular for dyeing keratin fibers with at least one direct dye, as well as a process using the same.

### BACKGROUND ART

It is known to dye keratin fibers, in particular human hair, with dyeing compositions containing oxidative coloring precursors, generally called oxidative bases, such as ortho- or para-phenylenediamines, ortho- or para-aminophenols and heterocyclic compounds. These oxidative bases are generally combined with couplers. These bases and these couplers are colorless or weakly colored compounds which, combined with oxidizing products, can give rise to colored compounds through an oxidative condensation process.

This type of coloring by oxidation makes it possible to obtain colors with very high visibility, coverage of white hair and in a wide variety of shades but it results in damage to the keratin fibers by the use of oxidizing agents and alkaline agents (in particular by repeated application or by combination with other hair treatments).

On the other hand, it is also known to dye keratin fibers, in particular human hair, with dyeing compositions containing direct dyes. Conventional direct dyes are in particular the following: benzene nitrates, anthraquinones, nitropyridines, azos, xanthines, acridines, azines, and triarylmethane type or natural colorings.

For example, JP-A-2002-241245 and WO 2009/047916 disclose a composition for dyeing hair, including a direct dye.

Hair coloration using direct dyes has advantages over hair coloration using oxidative dyes: it rarely cause allergic issues, no damage to the hair, and it gives vivid color visibility.

### DISCLOSURE OF INVENTION

However, skin staining has been an inevitable drawback of hair coloration using direct dyes.

In addition, in terms of usability, it is preferable that a composition for dyeing hair be a so-called one-part composition which is ready-to-use and therefore no mixing of two or more parts of the composition is necessary when coloring hair with the composition.

An objective of the present invention is to provide a composition for dyeing keratin fibers which uses a direct dye, but can prevent or reduce skin staining by the direct dye on the skin such as the scalp, while providing the keratin fibers with good cosmetic effects such as good coloring properties.

The above objective can be achieved by a composition for dyeing keratin fibers, comprising:
(a) at least one direct dye; and
(b) at least one oxygen-containing polycyclic compound which has at least one oxygen-containing polycyclic ring.

The (a) direct dye may be selected from the group consisting of acidic direct dyes, basic direct dyes and neutral direct dyes, and preferably from acidic direct dyes.

The (a) direct dye may be selected from the compounds according to the chemical formulae (II) and (II′), (III) and (III′), and (IV) shown later.

The amount of the (a) direct dye in the composition according to the present invention may range from 0.001% to 5% by weight, preferably from 0.01% to 3% by weight, and more preferably from 0.05% to 2% by weight, relative to the total weight of the composition.

The oxygen-containing polycyclic ring in the (b) oxygen-containing polycyclic compound may be selected from the group consisting of phthalan ring, coumaran ring, isobenzofuran ring, coumaron ring, chroman ring, chromen ring, isochromen ring, and piperonyl ring.

The oxygen-containing polycyclic ring of the (b) oxygen-containing polycyclic compound may have at least one substituent preferably selected from the group consisting of a hydroxyl group, an optionally substituted, linear or branched C₁₋₁₈ alkyl group, an optionally substituted, linear or branched C₁₋₁₈ alkoxy group, an optionally substituted C₃₋₁₈ alicyclic alkyl group, an optionally substituted, linear or branched C₂₋₁₈ alkenyl group, an optionally substituted, linear or branched C₂₋₁₈ alkenyloxy group, an optionally substituted, linear or branched C₂₋₁₈ alkynyl group, an optionally substituted, linear or branched C₂₋₁₈ alkynyloxy group, an optionally substituted C₆₋₁₈ aryl group, an optionally substituted C₇₋₁₈ aralkyl group, and an optionally substituted, linear or branched C₂₋₁₈ acyl group.

The (b) oxygen-containing polycyclic compound may be represented by the following chemical formula (1):

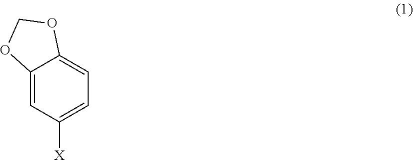

wherein
X denotes a hydroxyl, —(CH₂)ₙ—R or —(CH₂)ₙ—O—R; where n denotes an integer from 1 to 10, preferably 1 to 5, and more preferably 1 or 2, and R denotes a hydrogen, an optionally substituted, linear or branched C₁₋₁₈ alkyl group, an optionally substituted C₃₋₁₈ alicyclic alkyl group, an optionally substituted, linear or branched C₂₋₁₈ alkenyl group, an optionally substituted, linear or branched C₂₋₁₈ alkynyl group, an optionally substituted C₆₋₁₈ aryl group, an optionally substituted C₇₋₁₈ aralkyl group, and an optionally substituted, linear or branched C₂₋₁₈ acyl group.

The (b) oxygen-containing polycyclic compound may be piperonyl alcohol, heliotropyl acetone, or heliotropyl acetate, preferably piperonyl alcohol.

The amount of the (b) oxygen-containing polycyclic compound in the composition according to the present invention may range from 0.001% to 15% by weight, preferably from 0.01% to 10% by weight, and more preferably from 0.05% to 5% by weight, relative to the total weight of the composition.

The composition according to the present invention may further comprises (c) at least one sulfone compound represented by the following formula (I):

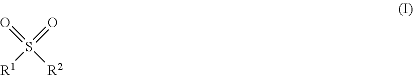

wherein
each of R¹ and R² independently denotes a monovalent C₁₋₃₀ aliphatic group or a monovalent C₆₋₃₀ aromatic group, which may optionally be substituted with at least one substituent; or
R¹ and R², together with the sulfur atom which they are binding, form a 3-10 membered ring which may optionally be substituted with at least one substituent.

In the above formula (I), the monovalent C₁₋₃₀ aliphatic group may be

a saturated monovalent C₁₋₃₀ aliphatic hydrocarbon group, preferably a linear or branched C₁₋₃₀ alkyl group, or a C₃₋₃₀ cycloalkyl group, or
an unsaturated monovalent C₂₋₃₀ aliphatic hydrocarbon group.

In the above formula (I), the monovalent C₆₋₃₀ aromatic group may be a monovalent C₆₋₃₀ aromatic hydrocarbon group. The monovalent C₆₋₃₀ aromatic hydrocarbon group may be a C₆₋₃₀ aryl group, or a linear or branched C₇₋₃₀ aralkyl group.

In the above formula (I), R¹ and R², together with the sulfur atom which they are binding, may form a 3-10 membered aliphatic ring which may optionally be substituted with at least one substituent.

The amount of the (c) sulfone compound in the composition according to the present invention may range from 0.5 to 30% by weight, preferably from 1 to 20% by weight, and more preferably from 2 to 10% by weight, relative to the total weight of the composition.

The composition according to the present invention may further comprise water.

The present invention also relates to a process for dyeing keratin fibers, comprising the step of applying the composition according to the present invention to the keratin fibers.

### BEST MODE FOR CARRYING OUT THE INVENTION

After diligent research, the inventors have discovered that it is possible to provide a composition, preferably a one-part composition, for dyeing keratin fibers which includes a direct dye, but can prevent or reduce skin staining by the direct dye on the skin such as the scalp, while providing the keratin fibers with good cosmetic effects such as good coloring properties.

Thus, the composition according to the present invention is intended for dyeing keratin fibers and comprises:
(a) at least one direct dye; and
(b) at least one oxygen-containing polycyclic compound which has at least one oxygen-containing polycyclic ring.

The composition according to the present invention is preferably a so-called one-part composition which is ready-to-use and therefore no mixing of two or more parts of the composition is necessary when coloring hair with the composition.

Hereafter, the composition according to the present invention will be described in a detailed manner.

### [Direct Dye]

The composition according to the present invention includes (a) at least one direct dye. Two or more direct dyes may be used in combination. Thus, a single type of direct dye or a combination of different types of direct dyes may be used.

A direct dye means a colored substance which does not require the use of an oxidizing agent in order to develop its color.

The direct dye may be a natural direct dye or a synthetic direct dye.

The expression “natural direct dye” is understood to mean any dye or dye precursor that is naturally occurring and is produced by extraction (and optionally purification) from a plant matrix or an animal such as an insect, optionally in the presence of natural compounds such as ash or ammonia.

As natural direct dyes, mention may be made of quinone dyes (such as lawsone and juglone), alizarin, purpurin, laccaic acid, carminic acid, kermesic acid, purpurogallin, protocatechaldehyde, indigoids such as indigo, sorghum, isatin, betanin, curcuminoids (such as curcumin), spinulosin, various types of chlorophyll and chlorophyllin, hematoxylin, hematein, brazilein, brazilin, safflower dyes (such as carthamin), flavonoids (such as rutin, quercetin, catechin, epicatechin, morin, apigenidin, and sandalwood), anthocyans (such as apigeninidin and apigenin), carotenoids, tannins, orceins, santalins and cochineal carmine.

It is also possible to use extracts or decoctions containing natural direct dye(s), in particular henna-based extracts, curcuma longa extract, sorghum leaf-sheath extract, haematoxylon campechianum extract, green tea extract, pine bark extract, cocoa extract, and logwood extract.

It is preferable that the natural direct dye be chosen from the group consisting of curcuminoids, santalins, chlorophyllin, haematoxylin, haematein, brazilein, brazilin, sorghum, laccaic acid, lawsone, juglone, alizarin, purpurin, carminic acid, kermesic acid, purpurogallin, protocatechaldehyde, indigoids, isatin, spinulosin, apigenin, orcein, betanin, flavonoids, anthocyans, and extracts or decoctions containing these compounds.

Alternatively, the natural direct dyes may be preferably chosen, for example, from hydroxylated quinones, indigoids, hydroxyflavones, santalins A and B, isatin and its derivatives, and brasilin and its hydroxylated derivative.

The hydroxylated quinones are preferably benzoquinones, naphthoquinones, and mono- or polyhydroxylated anthraquinones which are optionally substituted with groups such as alkyl, alkoxy, alkenyl, chloro, phenyl, hydroxyalkyl and carboxyl.

The naphthoquinones are preferably lawsone, juglone, flaviolin, naphthazarin, naphthopurpurin, lapachol, plumbagin, chloroplumbagin, droserone, shikonin, 2-hydroxy-3-methyl-1,4-naphthoquinone, 3,5-dihydroxy-1,4-naphthoquinone, 2,5-dihydroxy-1,4-naphthoquinone, 2-methoxy-5-hydroxy-1,4-naphthoquinone and 3-methoxy-5-hydroxy-1,4-naphthoquinone.

The benzoquinones are preferably spinulosin, atromentin, aurentioglyocladin, 2,5-dihydroxy-6-methylbenzoquinone, 2-hydroxy-3-methyl-6-methoxybenzoquinone, 2, 5-dihydroxy-3,6-diphenylbenzoquinone, 2,3-dimethyl-5-hydroxy-6-methoxybenzoquinone and 2,5-dihydroxy-6-isopropylbenzoquinone.

The anthraquinones are preferably alizarin, quinizarin, purpurin, carminic acid, chrysophanol, kermesic acid, rhein, aloe emodin, pseudopurpurin, quinizarincarboxylic acid, frangula emodin, 2-methylquinizarin, 1-hydroxyanthraquinone and 2-hydroxyanthraquinone.

The indigoids are preferably indigo, indirubin, isoindigo and Tyrian purple.

The hydroxyflavones are preferably quercetin and morin.

The expression “synthetic direct dye” is understood to mean any dye or dye precursor that is produced by chemical synthesis.

The direct dye can be selected from the group consisting of acidic (anionic) direct dyes, basic (cationic) direct dyes, and neutral (nonionic) direct dyes.

Non-limiting examples of syntheticdyes include (nonionic) neutral, anionic (acidic), and cationic (basic) dyes such as azo, methine, carbonyl, azine, nitro(hetero)aryl types or tri(hetero)arylmethane direct dyes, porphyrins and phthalocyanines, alone or as mixtures.

More particularly, the azo dyes comprise an —N═N— functional group, the two nitrogen atoms of which are not simultaneously involved in a ring. However, it is not ruled out for one of the two nitrogen atoms of the —N═N— sequence to be involved in a ring.

The dyes of the family of the methines are more particularly compounds comprising at least one sequence chosen from >C═C< and —N═C<, the two atoms of which are not simultaneously involved in a ring. However, it is specified that one of the nitrogen or carbon atoms of the sequences can be involved in a ring. More particularly, the dyes of this family result from compounds of the following types: true methine (comprising one or more of the above-mentioned —C═C— sequences); azomethine (comprising at least one or more —C═N-sequences) with, for example, the azacarbocyanines and their isomers, the diazacarbocyanines and their isomers, the tetraazacarbocyanines; mono- and diarylmethane; indoamines (or diphenylamines); indophenols; indoanilines.

As regards the dyes of the family of the carbonyls, mention may be made, for example, of synthetic dyes chosen from acridone, benzoquinone, anthraquinone, naphthoquinone, benzanthrone, anthranthrone, pyranthrone, pyrazolanthrone, pyrimidinoanthrone, flavanthrone, indanthrone, flavone, (iso)violanthrone, isoindolinone, benzimidazolone, isoquinolinone, anthrapyridone, pyrazoloquinazolone, perinone, quinacridone, quinophthalone, naphthalimide, anthrapyrimidine, diketopyrrolopyrrole or coumarin dyes.

As regards the dyes of the family of the cyclic azines, mention may in particular be made of azine, xanthene, thioxanthene, fluorindine, acridine, (di)oxazine, (di)thiazine or pyronine dyes.

The nitro(hetero)aromatic dyes are more particularly nitrobenzene or nitropyridine direct dyes.

As regards the dyes of porphyrin or phthalocyanine type, use may be made of cationic or noncationic compounds optionally comprising one or more metals or metal ions, such as, for example, alkali and alkaline earth metals, zinc and silicon.

Mention may be made, as examples of synthetic direct dyes which are particularly suitable, of nitrobenzene dyes, azo, azomethine or methine direct dyes, azacarbocyanines, such as tetraazacarbocyanines (tetraazapentamethines), quinone and in particular anthraquinone, naphthoquinone or benzoquinone direct dyes, or azine, xanthene, triarylmethane, indoamine, phthalocyanine and porphyrin direct dyes, alone or as mixtures. More preferably still, these synthetic direct dyes are chosen from nitrobenzene dyes, azo, azomethine or methine direct dyes and tetraazacarbocyanines (tetraazapentamethines); alone or as mixtures.

Mention may be made, among the azo, azomethine, methine or tetraazapentamethine direct dyes which can be used according to the invention, of the cationic dyes described in Patent Applications WO 95/15144, WO 95/01772 and EP 714 954; FR 2 189 006, FR 2 285 851, FR-2 140 205, EP 1 378 544 and EP 1 674 073.

Thus, mention may very particularly be made of the cationic direct dyes corresponding to the following formulae:

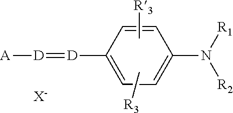

in which:
D represents a nitrogen atom or the —CH group,
R₁ and R₂, which are identical or different, represent a hydrogen atom; a C₁-C₄ alkyl radical which can be substituted by a —CN, —OH or —NH₂ radical or can form, with a carbon atom of the benzene ring, an optionally oxygen-comprising or nitrogen-comprising heterocycle which can be substituted by one or more C₁-C₄ alkyl radicals; or a 4′-aminophenyl radical,
R₃ and R′₃, which are identical or different, represent a hydrogen atom, a halogen atom chosen from chlorine, bromine, iodine and fluorine, a cyano radical, a C₁-C₄ alkyl radical, a C₁-C₄ alkoxy radical or an acetyloxy radical,
X⁻ represents an anion, preferably chosen from chloride, methyl sulphate and acetate,
A represents a group chosen from the following structures:

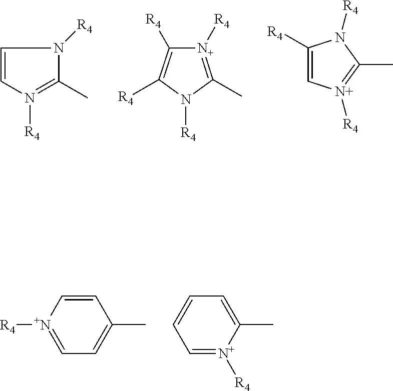

in which R₄ represents a C₁-C₄ alkyl radical which can be substituted by a hydroxyl radical;

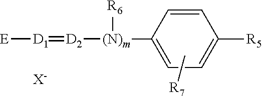

in which:
R₅ represents a hydrogen atom, a C₁-C₄ alkoxy radical or a halogen atom, such as bromine, chlorine, iodine or fluorine,
R₆ represents a hydrogen atom or a C₁-C₄ alkyl radical or forms, with a carbon atom in the benzene ring, a heterocycle which optionally comprises oxygen and/or is optionally substituted by one or more C₁-C₄ alkyl groups,
R₇ represents a hydrogen atom or a halogen atom, such as bromine, chlorine, iodine or fluorine,
D₁ and D₂, which are identical or different, represent a nitrogen atom or the —CH group, m=0 or 1,
X⁻ represents a cosmetically acceptable anion preferably chosen from chloride, methyl sulphate and acetate,
E represents a group chosen from the following structures:

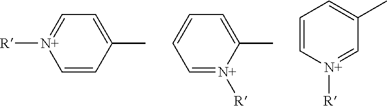

in which R′ represents a C₁-C₄ alkyl radical;
when m=0 and when D₁ represents a nitrogen atom, then E can also denote a group with the following structure:

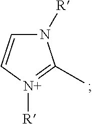

in which R′ represents a C₁-C₄ alkyl radical.

The synthetic direct dye may be selected from fluorescent dyes. Two or more types of fluorescent dyes may be used in combination.

The use of some fluorescent dyes may make it possible to obtain, on dark hair, colors which are more visible, than with conventional hydrophilic or hydrophobic direct dyes.

Furthermore, these fluorescent dyes, when applied to dark hair, may also make it possible to lighten the hair without damaging it.

As used herein, the term “fluorescent dyes” is understood to mean fluorescent compounds and optical brighteners. In at least one embodiment, the fluorescent dye is soluble in the medium of the composition.

Fluorescent dyes are fluorescent compounds which absorb visible radiation, for example, wavelengths ranging from 400 to 800 nm, and which are capable of re-emitting light in the visible region at a higher wavelength.

According to one embodiment, the fluorescent dyes useful for the present invention re-emit orange-colored fluorescent light. They exhibit, for instance, a maximum re-emission wavelength ranging from 500 to 700 nm.

Non-limiting examples of fluorescent dyes include compounds known in the art, for example, those described in Ullmann's Encyclopedia of Industrial Chemistry, Release 2004, 7th edition, “Fluorescent Dyes” chapter.

The optical brighteners of the present disclosure, also known under the name of “brighteners”, or “fluorescent brighteners”, or “fluorescent brightening agents” or “FWA”, or “fluorescent whitening agents”, or “whiteners”, or “fluorescent whiteners”, are colorless transparent compounds as they do not absorb in visible light but only in ultraviolet light (wavelengths ranging from 200 to 400 nanometers) and convert the energy absorbed into fluorescent light of higher wavelength emitted in the visible part of the spectrum, generally in the blue and/or green, that is to say in wavelengths ranging from 400 to 550 nanometers.

Optical brighteners are known in the art, for example, they are described in Ullmann's Encyclopedia of Industrial Chemistry (2002), “Optical Brighteners” and Kirk-Othmer Encyclopedia of Chemical Technology (1995): “Fluorescent Whitening Agents”.

The fluorescent dyes which can be used in the composition of the present disclosure include compounds known from the art, for example, those described in French Patent No. 2 830 189.

Soluble fluorescent compounds that may especially be mentioned include those belonging to the following families: naphthalimides, coumarins, xanthenes and in particular xanthenodiquinolizines and azaxanthenes; naphtholactams; azlactones; oxazines; thiazines; dioxazines; azo compounds; azomethines; methines; pyrazines; stilbenes; ketopyrroles; and pyrenes.

If present, the fluorescent dyes are preferred, more particularly, those re-emitting orange-colored fluorescent light.

In terms of ionic nature, the (a) direct dye may be selected from the group consisting of acidic direct dyes, basic direct dyes and neutral direct dyes, which covers all possible types of direct dyes, such as so-called nitro dyes and HC dyes. Acidic direct dyes have an anionic moiety in their chemical structure. Basic direct dyes have a cationic moiety in their chemical structure. Neutral direct dyes are nonionic.

According to an embodiment, it is preferable that the (a) direct dye be selected from acidic direct dyes.

The anionic direct dyes are commonly known as “acidic direct dyes” for their affinity with alkaline substances (see, for example, “Industrial Dyes., Chemistry, Properties, Application”, Klaus Hunger Ed. Wiley-VCH Verlag GmbH & Co KGaA, Weinheim 2003). Anionic or acid dyes are known in the literature (see, for example, “Ullman's Encyclopedia of Industrial Chemistry”, Azo Dyes, 2005 Wiley-VCH Verlag GmbH & Co. KGaA, Weinheim 10.1002/14356007.a03 245, point 3.2; ibid, Textile Auxiliaries, 2002 Wiley-VCH Verlag GmbH & Co. KGaA, Weinheim 10.1002/14356007.a26 227 and “Ashford's Dictionary of Industrial Chemicals”, Second Edition, p. 14-p. 39, 2001).

The term “anionic direct dyes” means any direct dye comprising in its structure at least one sulfonate group SO₃⁻ and/or at least one carboxylate group C(O)O⁻ and/or at least one phosphonate group P(═O)O⁻O⁻ and optionally one or more anionic groups G⁻ with G⁻, which may be identical or different, representing an anionic group chosen from alkoxide O⁻, thioalkoxide S⁻, phosphonate, carboxylate and thiocarboxylate: C(Q)Q′ with Q and Q′, which may be identical or different, representing an oxygen or sulfur atom; preferably, G⁻ represents a carboxylate, i.e. Q and Q′ represent an oxygen atom.

The preferred anionic dyes of formula of the invention are chosen from acidic nitro direct dyes, acidic azo dyes, acidic azine dyes, acidic triarylmethane dyes, acidic indoamine dyes, acidic anthraquinone dyes, anionic styryl dyes, and indigoids and acidic natural dyes; each of these dyes containing at least one sulfonate, phosphonate or carboxylate group bearing a cationic counterion X⁺, where X⁺ represents an organic or mineral cationic counter ion preferably chosen from alkali and alkaline-earth metals, such as Na⁺ and K⁺

Preferred acid dyes may be chosen from:
a) the diaryl anionic azo dyes of formula (II) or (II′):

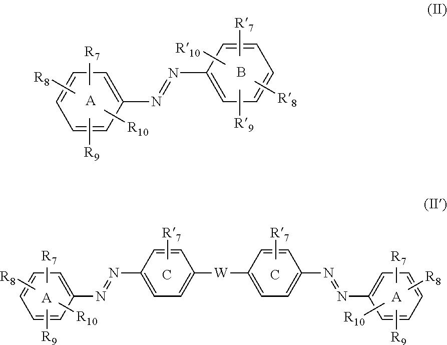

in which formulae (II) and (II′):
R₇, R₈, R₉, R₁₀, R′₇, R′₈, R′₉ and R′₁₀, which may be identical or different, represent a hydrogen atom or a group chosen from:
   alkyl;
   alkoxy, alkylthio;
   hydroxyl, mercapto;
   nitro;
   R^{º}—C(X)—X′—, R^{º}—X′—C(X)—, R^{º}—X′—C(X)—X″— with R^{º} representing a hydrogen atom or an alkyl or aryl group; X, X′ and X″, which may be identical or different, representing an oxygen or sulfur atom, or NR with R representing a hydrogen atom or an alkyl group;
   (O)₂S(O⁻)—, X⁺ as defined previously;
   (O)CO⁻—, X⁺ as defined previously;
   (O)P(O₂⁻)—, 2X⁺ as defined previously;
   R″—S(O)₂—, with R″ representing a hydrogen atom or an alkyl, aryl, (di)(alkyl)amino or aryl(alkyl)amino group; preferably a phenylamino or phenyl group;
   R′″—S(O)₂—X′— with R′″ representing an alkyl or optionally substituted aryl group, X′ as defined previously;
   (di)(alkyl)amino;
   aryl(alkyl)amino optionally substituted with one or more groups chosen from i) nitro; ii) nitroso; iii) (O)₂S(O⁻)—, X⁺ and iv) alkoxy with X⁺;
   optionally substituted heteroaryl; preferably a benzothiazolyl group;
   cycloalkyl; especially cyclohexyl,
   Ar—N═N— with Ar representing an optionally substituted aryl group, preferably a phenyl optionally substituted with one or more alkyl,
   (O)₂S(O⁻)—, X⁺ or phenylamino groups;
   or alternatively two contiguous groups R₇ with R or R with R₉ or R₉ with R₁₀ together form a fused benzo group A′; and R′₇ with R′₈ or R′₈ with R′₉ or R′₉ with R′₁₀ together form a fused benzo group B′; with A′ and B′ optionally substituted with one or more groups chosen from i) nitro; ii) nitroso; iii) (O)₂S(O⁻)—, X⁺; iv) hydroxyl; v) mercapto; vi) (di)(alkyl)amino; vii) R^{º}—C(X)—X′—; viii) R^{º}—X′—C(X)—; ix) R^{º}—X′—C(X)—X″—; x) Ar—N═N— and xi) optionally substituted aryl(alkyl)amino; with X⁺, R^{º}, X, X′, X″ and Ar as defined previously;
W represents a sigma bond σ, an oxygen or sulfur atom, or a divalent radical i) —NR— with R as defined previously, or ii) methylene —C(Rₐ)(R_{b})— with Rₐ and R_{b}, which may be identical or different, representing a hydrogen atom or an aryl group, or alternatively Rₐ and R_{b} form, together with the carbon atom that bears them, a spiro cycloalkyl; preferably W represents a sulfur atom or Rₐ and R_{b} together form a cyclohexyl;
   it being understood that formulae (II) and (II′) comprise at least one sulfonate (O)₂S(O⁻)—, X⁺ or phosphonate (O)P(O₂⁻) 2X⁺ or carboxylate (O)C(O⁻)—, X⁺ radical on one of the rings A, A′, B, B′ or C with X⁺ as defined previously;

As examples of dyes of formula (II), mention may be made of Acid Red 1, Acid Red 4, Acid Red 13, Acid Red 14, Acid Red 18, Acid Red 27, Acid Red 32, Acid Red 33, Acid Red 35, Acid Red 37, Acid Red 40, Acid Red 41, Acid Red 42, Acid Red 44, Acid Red 68, Acid Red 73, Acid Red 135, Acid Red 138, Acid Red 184, Food Red 1, Food Red 13, Food Red 17, Acid Orange 6, Acid Orange 7, Acid Orange 10, Acid Orange 19, Acid Orange 20, Acid Orange 24, Acid Yellow 9, Acid Yellow 36, Acid Yellow 199, Food Yellow 3; Acid Violet 7, Acid Violet 14, Acid Blue 113, Acid Blue 117, Acid Black 1, Acid Brown 4, Acid Brown 20, Acid Black 26, Acid Black 52, Food Black 1, Food Black 2, Pigment Red 57;

and as examples of dyes of formula (II′), mention may be made of Acid Red 111, Acid Red 134, Acid yellow 38;
b) the anthraquinone dyes of formulae (III) and (III′):

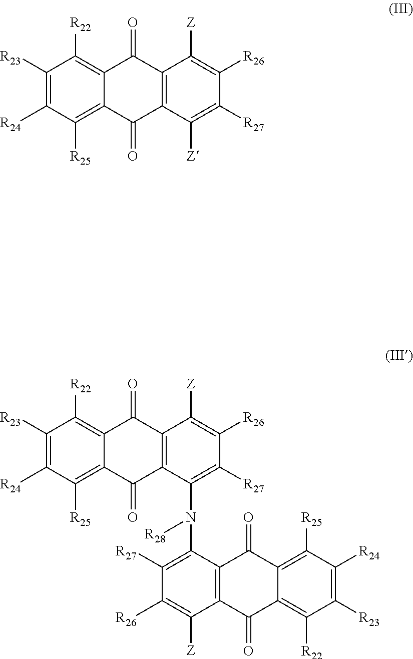

in which formulae (III) and (III′):
R₂₂, R₂₃, R₂₄, R₂₅, R₂₆ and R₂₇, which may be identical or different, represent a hydrogen or halogen atom or a group chosen from:
   alkyl;
   hydroxyl, mercapto;
   alkoxy, alkylthio;
   aryloxy or arylthio optionally substituted, preferably substituted with one or more groups chosen from alkyl and (O)₂S(O⁻)—, X⁺ with X⁺ as defined previously;
   aryl(alkyl)amino optionally substituted with one or more groups chosen from alkyl and (O)₂S(O⁻)—, X⁺ with X⁺ as defined previously;
   (di)(alkyl)amino;
   (di)(hydroxyalkyl)amino;
   (O)₂S(O⁻)—, X⁺ with X⁺ as defined previously;
Z′ represents a hydrogen atom or a group NR₂₈R₂₉ with R₂₈ and R₂₉, which may be identical or different, representing a hydrogen atom or a group chosen from:
   alkyl;
   polyhydroxyalkyl such as hydroxyethyl;
   aryl optionally substituted with one or more groups, particularly i) alkyl such as methyl, n-dodecyl, n-butyl; ii) (O)₂S(O⁻)—, X with X⁺ as defined previously; iii) R^{º}—C(X)—X′—, R^{º}—X′—C(X)—, R^{º}—X′—C(X)—X″— with R^{º}, X, X′ and X″ as defined previously, preferably R^{º} represents an alkyl group;
   cycloakyl; especially cyclohexyl;
Z represents a group chosen from hydroxyl and NR′₂₈R′₂₉ with R′₂₈ and R′₂₉, which may be identical or different, representing the same atoms or groups as R₂₈ and R₂₉ as defined previously;
   it being understood that formulae (III) and (III′) comprise at least one sulfonate group (O)₂S(O⁻)—, X⁺ with X⁺ as defined previously;

As examples of dyes of formula (III), mention may be made of Acid Blue 25, Acid Blue 43, Acid Blue 62, Acid Blue 78, Acid Blue 129, Acid Blue 138, Acid Blue 140, Acid Blue 251, Acid Green 25, Acid Green 41, Acid Violet 42, Acid Violet 43, Mordant Red 3; EXT Violet 2, and as examples of dyes of formula (III′), mention may be made of Acid Black 48; and
g) the quinoline-based dyes of formula (IV):

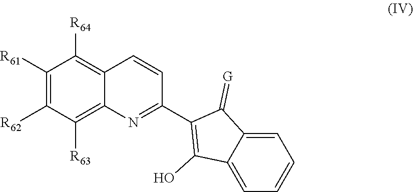

in which formula (IV):
R₆₁ represents a hydrogen or halogen atom or an alkyl group;
R₆₂, R₆₃ and R₆₄, which may be identical or different, represent a hydrogen atom or a group (O)₂S(O⁻)—, X⁺ with X⁺ as defined previously;
or alternatively R₆₁ with R₆₂, or R₆₁ with R₆₄, together form a benzo group optionally substituted with one or more groups (O)₂S(O⁻)—, X⁺ with X⁺ as defined previously;
G represents an oxygen or sulfur atom or a group NRₑ with Rₑ representing a hydrogen atom or an alkyl group; particularly G represents an oxygen atom;
   it being understood that formula (IV) comprises at least one sulfonate group (O)₂S(O⁻)—, X⁺ with X⁺ as defined previously;

As examples of dyes of formula (IV), mention may be made of Acid Yellow 2, Acid Yellow 3 and Acid Yellow 5.

It is preferable that the acidic direct dye be selected from the group consisting of Yellow 5, Orange 4, EXT. Violet 2 and Acid Black 1.

The composition according to the present invention may contain the (a) direct dye(s) in an amount of from 0.001% to 5% by weight, preferably from 0.01 to 3% by weight, and more preferably 0.05 to 2% by weight, relative to the total weight of the composition.

### [Oxygen-Containing Polycyclic Compound]

The composition according to the present invention includes (b) at least one oxygen-containing polycyclic compound. Two or more oxygen-containing polycyclic compounds may be used in combination. Thus, a single type of oxygen-containing polycyclic compound or a combination of different types of oxygen-containing polycyclic compounds may be used.

The (b) oxygen-containing polycyclic compound has at least one oxygen-containing polycyclic ring. Thus, the (b) oxygen-containing polycyclic compound may have a plurality of oxygen-containing polycyclic rings. It is preferable that the (b) oxygen containing polycyclic compound has one oxygen-containing polycyclic ring.

The oxygen-containing polycyclic ring in the (b) oxygen-containing polycyclic compound is not limited. It is preferable that the oxygen-containing polycyclic ring comprise an aromatic ring and preferably comprises at least one benzene ring. The oxygen-containing polycyclic ring may comprise preferably 6-30 membered ring, more preferably 8-10 membered ring, and more preferably 9-10 membered ring. The oxygen-containing polycyclic ring comprises preferably 1-3 oxygen atoms, and more preferably 1 or 2 oxygen atoms.

It is preferable that the oxygen-containing polycyclic ring be selected from the group consisting of

phthalan ring (1,3-dihydroisobenzofuran) represented by the chemical formula

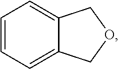

coumaran ring (2,3-dihydrobenzofuran) represented by the chemical formula

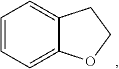

isobenzofuran ring represented by the chemical formula

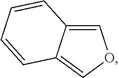

coumaron ring (benzofuran) represented by the chemical formula

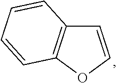

chroman ring (3,4-dihydro-2H-1 benzopyran) represented by the chemical formula

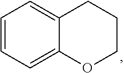

chromen ring (2-benzopyran and 4-benzopyran) represented by the chemical formulae

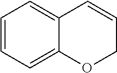

(2-benzopyran)

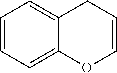

(4-benzopyran isochromen ring (1H-isochromen and 3H-isochromen) represented by the chemical formulae

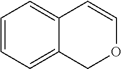

### (1H-isochromen)

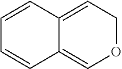

(3H-isochromen), and piperonyl ring (1,2-methyleneioxybenzene) represented by the chemical formula

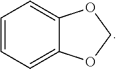

It is preferable that the oxygen-containing polycyclic ring of the (b) oxygen-containing polycyclic compound have at least one substituent (substituent A) preferably selected from the group consisting of a hydroxyl group, an optionally substituted, linear or branched C₁₋₁₈ alkyl group, an optionally substituted, linear or branched C₁₋₁₈ alkoxy group, an optionally substituted C₃₋₁₈ alicyclic alkyl group, an optionally substituted, linear or branched C₂₋₁₈ alkenyl group, an optionally substituted, linear or branched C₂₋₁₈ alkenyloxy group, an optionally substituted, linear or branched C₂₋₁₈ alkynyl group, an optionally substituted, linear or branched C₂₋₁₈ alkynyloxy group, an optionally substituted C₆₋₁₈ aryl group, an optionally substituted C₇₋₁₈ aralkyl group, and an optionally substituted, linear or branched C₂₋₁₈ acyl group.

It is more preferable that the (b) oxygen-containing polycyclic compound comprise an oxygen-containing polycyclic ring, preferably selected from the group consisting of phthalan ring, coumaran ring, isobenzofuran ring, coumaron ring, chroman ring, chromen ring, isochromen ring, and piperonyl ring, and even more preferably piperonyl ring, which is substituted with at least one substituent A as mentioned above.

As examples of the substituent (substituent B) for the substituent A, mention may be made of a monovalent functional group such as a halogen atom, a hydroxyl group, a C₁-C₆ alkoxy group, an amino group, a C₁-C₆ alkylamino group, a C₁-C₆ dialkylamino group, a nitro group, an aldehyde group, a C₂-C₆ acyl group, a carboxyl group, a cyano group and the like. As the substituent B, a hydroxyl group is preferable.

Thus, the substituent A is preferably selected from the group consisting of a hydroxyl group, a linear or branched C₁₋₁₈ alkyl group substituted with at least one hydroxyl group, a C₃₋₁₈ alicyclic alkyl group substituted with at least one hydroxyl group, a linear or branched C₂₋₁₈ alkenyl group substituted with at least one hydroxyl group, a linear or branched C₂₋₁₈ alkynyl group substituted with at least one hydroxyl group, a C₆₋₁₈ aryl group substituted with at least one hydroxyl group, a C₇₋₁₈ aralkyl group substituted with at least one hydroxyl group, and a linear or branched C₂₋₁₈ acyl group substituted with at least one hydroxyl group, which may optionally be further substituted with at least one substituent B other than a hydroxyl group.

The substituent A is more preferably a hydroxyl group or a linear or branched C₁₋₆ alkyl group substituted with at least one hydroxyl group, and even more preferably one hydroxyl group. Most preferably the substituent A is a hydroxyl methyl group or a hydroxyl ethyl group.

As examples of the substituent B other than a hydroxyl group, mention may be made of a monovalent functional group such as a halogen atom, a C₁-C₆ alkoxy group, an amino group, a C₁-C₆ alkylamino group, a C₁-C₆ dialkylamino group, a nitro group, an aldehyde group, a C₂-C₆ acyl group, a carboxyl group, a cyano group and the like.

It is preferable that the (b) oxygen-containing polycyclic compound be represented by the following chemical formula (1):

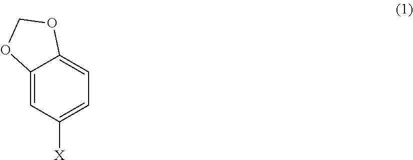

wherein
X denotes a hydroxyl, —(CH₂)ₙ—R or —(CH₂)ₙ—O—R; where n denotes an integer from 1 to 10, preferably 1 to 5, and more preferably 1 or 2, and R denotes a hydrogen, an optionally substituted, linear or branched C₁₋₁₈ alkyl group, an optionally substituted C₃₋₁₈ alicyclic alkyl group, an optionally substituted, linear or branched C₂₋₁₈ alkenyl group, an optionally substituted, linear or branched C₂₋₁₈ alkynyl group, an optionally substituted C₆₋₁₈ aryl group, an optionally substituted C₇₋₁₈ aralkyl group, or an optionally substituted, linear or branched C₂₋₁₈ acyl group.

As examples of the substituent for the group X, mention may be made of a monovalent functional group such as a halogen atom, a hydroxyl group, a C₁-C₆ alkoxy group, an amino group, a C₁-C₆ alkylamino group, a C₁-C₆ dialkylamino group, a nitro group, an aldehyde group, a C₂-C₆ acyl group, a carboxyl group, a cyano group and the like. As the substituent for the group X, a hydroxyl group is preferable.

It is preferable that R denote a hydrogen, an optionally substituted, linear or branched C₁₋₁₈ alkyl group, an optionally substituted C₃₋₁₈ alicyclic alkyl group, an optionally substituted, linear or branched C₂₋₁₈ alkenyl group, or an optionally substituted, linear or branched C₂₋₁₈ acyl group.

As specific examples of a linear or branched C₁₋₁₈ alkyl group, mention may be made of a straight chain alkyl group such as methyl group, ethyl group, n-propyl group, n-butyl group, n-hexyl group, n-octyl group, n-nonyl group, n-decyl group, n-undecyl group, n-dodecyl group, n-tetradecyl group, n-pentadecyl group, hexadecyl group, octadecyl group and the like; and a branched chain alkyl group such as isopropyl group, isobutyl group, sec-butyl group, tert-butyl group, 4-methylpentyl group, 5-methylhexyl group, 2-ethylhexyl group, 6-methylheptyl group, 7-methyloctyl group, 8-methylnonyl group, 2,6-dimethylheptyl group, 3,7-dimethyloctyl group, 3,7,11-trimethyldodecyl group and the like. However, specific examples are not restricted thereto.

As specific examples of a C₃₋₁₈ alicyclic alkyl group, mention may be made of cyclopropyl group, cyclobutyl group, cyclopentyl group, cyclohexyl group and cycloheptyl group with cyclohexyl group being preferred. However, specific examples are not restricted thereto.

As other specific examples of a C₃₋₁₈ alicyclic alkyl group, mention may be made of 4-isopropylcyclohexyl group, 4-tert-butylcyclohexyl group, 2,4-dimethyl-3-cyclohexenyl group, 2-tert-butylcyclohexyl group, 4-isopropylcyclohexylmethyl group, 5-methyl-2-(1-methylethenyl)cyclohexyl group (isopulegyl), 5-methyl-2-isopropylcyclohexyl group (p-menthan-3-yl, menthyl), 1-methyl, 4-isopropylcyclohexenyl group (terpinenyl), 1-methyl-4-isopropylcyclohexyl group (dihydroterpinenyl), 1-methyl-4-isopropenyl-6-cyclohexen-2-yl group (carvenyl), 6-methyl-3-isopropenylcyclohexenyl group (dihydrocarvenyl), 1-(4-isopropenyl)cyclohexyl)methyl group (perillyl), 4-methyl-1-isopropylbicyclo[3.1.0]hexan-4-yl group (4-thujanyl), 4-methyl-1-isopropylbicyclo[3.1.0]hexan-3-yl group (3-thujanyl), 6,6-dimethylbicyclo[3.1.1]hept-2-en-2-ethyl group (nopyl), 1,3,3-trimethylbicyclo[2.2.1]heptan-2-yl group (fenchonyl) and endo-1,7,7-trimethylbicyclo[2.2.1]heptan-2-yl group (bornyl).

As specific examples of a linear or branched C₂₋₁₈ alkenyl group, mention may be made of a straight chain alkenyl group such as vinyl group, allyl group, 1-propenyl group, 1-butenyl group, 2-butenyl group, 3-butenyl group, 4-pentenyl group, 5-hexenyl group, 6-heptenyl group, 7-octenyl group, 8-nonenyl group, 9-decenyl group, 1-pentenyl group, 1-hexenyl group, 1-heptenyl group, 1-octenyl group, 1-nonenyl group, 1-decenyl group, 9-octadecenyl group and the like; and a branched chain alkenyl group such as isopropenyl group, 2-methyl-1-propenyl group, 3-methyl-2-butenyl group, 4-methyl-3-pentenyl group, 5-methyl-4-hexenyl group, 6-methyl-5-heptenyl group, 7-methyl-6-octenyl group, 8-methyl-7-noneyl group, 1-methyl-1-propenyl group, 1-methyl-1-butenyl group, 1-methyl-1-pentenyl group, 1-methyl-1-hexenyl group, 1-methyl-1-heptenyl group, 1-methyl-1-octenyl group, 1-methyl-1-nonenyl group, 2,6-dimethyl-5-hetenyl group, 2,6-dimethyl-1-heptenyl group, 3,7-dimethyl-2,6-octadienyl (geranyl, neryl) group, 3,7,11-trimethyl-2,6,10-dodecatrienyl (farnesyl) group, 3,7,11-trimethyl-6,10-dodecadienyl 50 (dihydrofarnesyl) group and the like. However, specific examples are not restricted thereto. As specific examples of a linear or branched C₂₋₁₈ alkynyl group, mention may be made of ethynyl group, propynyl group, butynyl group, and the like. However, the specific examples are not restricted thereto.

As specific examples of a C₆₋₁₈ aryl group, mention may be made of a phenyl group, a tolyl group, a xylyl group, a naphtyl group and the like. However, the specific examples are not restricted thereto.

As specific examples of a C₇₋₁₈ aralkyl group, mention may be made of a benzyl, a phenethyl group, a (1-naphtyl)methyl group, (1-naphtyl)ethyl group, and the like. However, the specific examples are not restricted thereto.

As specific examples of a linear or branched C₂₋₁₈ acyl group, mention may be made of acetyl group (CH₃—CO—), propanoyl group, propenoyl group, and benzoyl group.

It is preferable that X be a hydroxyl group, or a linear or branched C₁₋₁₈ alkyl group, more preferably C₁₋₆ alkyl group, substituted with at least one hydroxyl group, preferably one hydroxyl group. Most preferably, X is a hydroxyl methyl group or a hydroxyl ethyl group.

As specific examples encompassed by the above formula (1) wherein X in the above formula (1) denotes —(CH₂)ₙ—O—R where n is as explained above, mention may be made of,

when R of the above formula (1) is an optionally substituted, linear or branched C₁₋₁₈ alkyl or C₂₋₁₈ alkenyl group, 3,4-methylenedioxybenzyl n-propyl ether (piperonyl n-propyl ether), 3,4-methylenedioxybenzyl n-butyl ether (piperonyl n-butyl ether), 3,4-methylenedioxybenzyl n-hexyl ether (piperonyl n-hexyl ether), 3,4-methylenedioxybenzyl 2-ethylhexyl ether (piperonyl 2-ethylhexyl ether), 3,4-methylenedioxybenzyl 3,7-dimethyl-2,6-octadienyl ether (piperonyl geranyl ether), 3,4-methylenedioxybenzyl 3,7,11-trimethyl-2,6,10-dodecatrienyl ether (piperonyl farnesyl ether); and
when R is an optionally substituted C₃₋₁₈ alicyclic alkyl group, 3,4-methylenedioxybenyl cyclohexyl ether (piperonyl cyclohexyl ether), 3,4-methylenedioxybenzyl 5-methyl-2-(1-methylethenyl)cyclohexyl ether (piperonyl isopulegyl ether), 3,4-methylenedioxybenzyl 5-methyl-2-isopropylcyclohexyl ether (piperonyl menthyl ether), 3,4-methylenedioxybenzyl 1-methyl-4-isopropylcyclohexenyl ether (piperonyl terpinenyl ether), 3,4-methylenedioxybenzyl 1-methyl-4-isopropylcyclohexyl ether (piperonyl dihydroterpinenyl ether), 3,4-methylenedioxybenzyl 1-methyl-4-isopropenyl-6-cyclohexen-2-yl ether (piperonyl carvenyl ether), 3,4-methylenedioxybenzyl 6-methyl-3-isopropenylcyclohexyl ether (piperonyl dihydrocarvenylether), 3,4-methylenedioxybenzyl [1-(4-isopropenyl)cyclohexyl]methyl ether (piperonyl perillyl ether), 3,4-methylenedioxybenzyl 4-methyl-1-isopropylbicyclo[3.1.0]hexan-4-yl ether (piperonyl 4-thujanyl ether), 3,4-methylenedioxybenzyl 4-methyl-1-isopropylbicyclo[3.1.0]hexan-3-yl ether (piperonyl 3-thujanyl ether), 3,4-methylenedioxybenzyl 6,6-dimethylbicyclo[3.1.1]hept-2-en-2-ethyl ether (piperonyl nopyl ether), 3,4-methylenedioxybenzyl 1,3,3-trimethylbicyclo[2.2.1]heptan-2-yl ether (piperonyl fenchonyl ether) and 3,4-methylenedioxybenzyl endo-1,7,7-trimethylbicyclo[2.2.1]heptan-2-yl ether (piperonyl bornyl ether).

It is more preferable that X in formula (1) denote a hydroxyl (sesamol).

It is more preferable that, when X in formula (1) denotes —(CH₂)ₙ—O—R where n and R are as explained above, R denote a hydrogen (piperonyl alcohol) or a C₂₋₁₈ acyl group, preferably a C₂₋₆ acyl group, which may optionally be substituted with at least one substituent, and more preferably CH₃—CO— (heliotropylacetate)

It is more preferable that, when X in formula (1) denotes —(CH₂)ₙ—R where n and R are as explained above, R denote a C₂₋₁₈ acyl group, preferably a C₂₋₆ acyl group, which may optionally be substituted with at least one substituent, and more preferably CH₃—CO— (heliotropylacetone).

It is even more preferable that the (b) oxygen-containing polycyclic compound be piperonyl alcohol, sesamol, heliotropyl acetone, or heliotropyl acetate, in particular piperonyl alcohol.

The composition according to the present invention may contain the oxygen-containing polycyclic compound in an amount of from 0.001% to 15% by weight, preferably from 0.01% to 10% by weight, and more preferably from 0.05% to 5% by weight, relative to the total weight of the composition.

### [Sulfone Compound]

It is preferable that the composition according to the present invention further include (c) at least one specific sulfone compound. Two or more specific sulfone compounds may be used in combination. Thus, a single type of specific sulfone compound or a combination of different types of sulfone compounds may be used.

The (c) sulfone compound used in the present invention can be represented by the following formula (I):

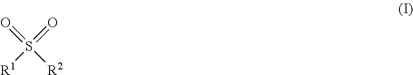

wherein
each of R¹ and R² independently denotes a monovalent C₁₋₃₀, preferably C₁₋₂₀, more preferably C₁₋₁₀ aliphatic group or a monovalent C₆₋₃₀, preferably C₆₋₂₀, more preferably C₆₋₁₀ aromatic group, which may optionally be substituted with at least one substituent; or R¹ and R², together with the sulfur atom which they are binding, form a 3-10, preferably 4-10, and more preferably 4-8 membered ring which may optionally be substituted with at least one substituent.

In the above formula (I), the monovalent C₁₋₃₀, preferably C₁₋₂₀, and more preferably C₁₋₁₀ aliphatic group may be

a saturated monovalent C₁₋₃₀, preferably C₁₋₂₀, and more preferably C₁₋₁₀ aliphatic hydrocarbon group, preferably a linear or branched C₁₋₃₀, preferably C₁₋₂₀, and more preferably C₁₋₁₀ alkyl group, or a C₃₋₃₀, preferably C₄₋₂₀, and more preferably C₅₋₁₀ cycloalkyl group, or
an unsaturated monovalent C₂₋₃₀, preferably C₂₋₂₀, and more preferably C₂₋₁₀ aliphatic hydrocarbon group.

As examples of a linear or branched C₁₋₃₀, preferably C₁₋₂₀, and more preferably C₁₋₁₀ alkyl group, mention may be made of a methyl group, an ethyl group, an n-propyl group, an isopropyl group, an n-butyl group, an isobutyl group, a sec-butyl group, a tert-butyl group, a pentyl group, a hexyl group, a heptyl group, an octyl group, a nonyl group, a decyl group, and the like.

As examples of a C₃₋₃₀, preferably C₄₋₂₀, and more preferably C₅₋₁₀ cycloalkyl group, mention may be made of a cyclopropyl group, a cyclobutyl group, a cyclopentyl group, a cyclohexyl group, a cycloheptyl group, a cyclooctyl group, and the like.

As examples of an unsaturated monovalent C₂₋₃₀, preferably C₂₋₂₀, and more preferably C₂₋₁₀ aliphatic hydrocarbon group, mention may be made of a linear or branched C₂₋₃₀, preferably C₂₋₂₀, and more preferably C₂₋₁₀ alkenyl group such as a vinyl group, a 1-propenyl group, an allyl group, an isopropenyl group, a 1-butenyl group, a 2-butenyl group, a pentenyl group, a hexenyl group, and the like; as well as, a C₂₋₃₀, preferably C₂₋₂₀, and more preferably C₂₋₁₀ cycloalkenyl group such as a cyclopentenyl group, a cyclohexenyl group, and the like.

In the above formula (I), the monovalent C₆₋₃₀, preferably C₆₋₂₀, and more preferably C₆₋₁₀ aromatic group may be a monovalent C₆₋₃₀, preferably C₆₋₂₀, and more preferably C₆₋₁₀ aromatic hydrocarbon group. The term “aromatic group” here means a group including at least one aromatic moiety. Thus, the monovalent C₆₋₃₀, preferably C₆₋₂₀, and more preferably C₆₋₁₀ aromatic group may be a C₆₋₃₀, preferably C₆₋₂₀, and more preferably C₆₋₁₀ aryl group, or a linear or branched C₇₋₃₀, preferably C₇₋₂₀, and more preferably C₇₋₁₀ aralkyl group.

As examples of a C₆₋₃₀, preferably C₆₋₂₀, and more preferably C₆₋₁₀ aryl group, mention may be made of a phenyl group, a tolyl group, a xylyl group, a mesityl group, and the like.

As examples of a linear or branched C₇₋₃₀, preferably C₇₋₂₀, and more preferably C₇₋₁₀ aralkyl group, mention may be made of a benzyl group, a phenethyl group and the like.

In the above formula (I), R¹ and R², together with the sulfur atom which they are binding, may form a 3-10, preferably 4-10, and more preferably 4-8 membered aliphatic ring which may optionally be substituted with at least one substituent.

The 3-10, preferably 4-10, and more preferably 4-8 membered aliphatic ring may be formed by a C₃₋₁₀, preferably C₄₋₁₀, and more preferably C₄₋₈ divalent, linear or branched, saturated or unsaturated hydrocarbon group, which binds to the sulfur atom.

As examples of a C₃₋₁₀, preferably C₄₋₁₀, and more preferably C₄₋₈ divalent, linear or branched, saturated hydrocarbon group, mention may be made of a C₃₋₁₀, preferably C₄₋₁₀, and more preferably C₄₋₈ alkylene group such as a propylene group, an isopropylene (—CH₂—CH(CH₃)—) group, an n-butylene group, an isobutylene (—CH₂—CH(CH₃)—CH₂—) group, a sec-butylene (—CH(CH₃)—CH₂—CH₂—) group, a tert-butylene (—CH₂—C(CH₃)₂-) group, a pentylene group, a hexylene group, and the like.

As examples of a C₃₋₁₀, preferably C₄₋₁₀, and more preferably C₄₋₈ divalent, linear or branched, unsaturated hydrocarbon group, mention may be made of a C₃₋₁₀, preferably C₄₋₁₀, and more preferably C₄₋₈ alkenylene group such as a propenylene group, 1-butenylene, 2-butenylene group and the like.

As examples of the substituent which may be present on the monovalent C₁₋₃₀, preferably C₁₋₂₀, and more preferably C₁₋₁₀ aliphatic or a monovalent C₆₋₃₀, preferably C₆₋₂₀, and more preferably C₆₋₁₀ aromatic group, as R¹ or R², or on the 3-10, preferably 4-10, and more preferably 4-8 membered ring formed by R¹ and R² together with the sulfur atom which they are binding, mention may be made of a monovalent functional group such as a halogen atom, a hydroxyl group, a C₁-C₆ alkoxy group, an amino group, a C₁-C₆ alkylamino group, a C₁-C₆ dialkylamino group, a nitro group, a carbonyl group, an acyl group, a carboxyl group, a cyano group and the like.

The monovalent C₁₋₃₀, preferably C₁₋₂₀, and more preferably C₁₋₁₀ aliphatic or a monovalent C₆₋₃₀, preferably C₆₋₂₀, and more preferably C₆₋₁₀ aromatic group, as R¹ or R², or the 3-10, preferably 4-10, and more preferably 4-8 membered ring formed by R¹ and R² together with the sulfur atom which they are binding, may optionally contain at least one heteroatom selected from the group consisting of an oxygen, nitrogen and sulfur atom, in the above substituent or in the main hydrocarbon chain of the group or the ring.

As specific examples of the (c) sulfone compound used in the present invention, mention may be made of dimethyl sulfone, methyl ethyl sulfone, diethyl sulfone, methyl isopropyl sulfone, ethyl isopropyl sulfone, diisopropylsulfone, 2-chloroethyl ethyl sulfone, di-n-butylsulfone, divinylsulfone; diphenyl sulfone, bis(4-hydroxyphenyl)sulfone, bis(4-aminophenyl)sulfone, bis(3-aminophenylsulfone), bis(4-chlorophenylsulfone), bis(4-fluorophenyl)sulfone, 2-hydroxyphenyl-4-hydroxyphenyl sulfone, phenyl-4-chlorophenyl sulfone, phenyl-2-aminophenyl sulfone, bis(3-amino-4-hydroxyphenyl)sulfone, dibenzylsulfone; and the like.

If R¹ and R², together with the sulfur atom which they are binding, form a 3-10, preferably 4-10, and more preferably 4-8 membered ring which may optionally be substituted with at least one substituent, the (c) compound may be substituted or non-substituted sulforane or sulforene. As the substituent, mention may be made of a monovalent functional group such as a monovalent C₁₋₃₀ aliphatic group as explained above, a halogen atom a such as a fluorine atom, a C₁-C₆ alkoxy group, an amino group, a C₁-C₆ alkylamino group, a C₁-C₆ dialkylamino group, a nitro group, an aldehyde group, a C₂-C₆ acyl group, a carboxyl group, and a cyano group. At least one hydrogen atom of the substituent, if present, may be replaced with a halogen atom a such as a fluorine atom. As specific examples of the substituted or non-substituted sulforane or sulforene, mention may be made of, 2-methyl-sulforane or sulforene, 3-methylsulforane or sulforene, 2,2-dimethyl-sylforane sulforene, 2,4-dimethyl-sulforane or sulforene, 2-fluoro-sulforane or sulforene, 3-fluoro-sulforane or sulforene, 2,2-difluoro-sulforane or sulforene, 2-trifluoromethyl-slforane or sulforene, 3-perfluoroethyl-sulforane or sulforene.

Since the composition according to the present invention includes the (c) sulfone compound(s), it can prevent or reduce skin staining by the (a) direct dye(s) on the skin such as the scalp.

The amount of the (c) sulfone compound(s) in the composition according to the present invention may range from 0.5 to 30% by weight, preferably from 1 to 20% by weight, and more preferably from 2 to 10% by weight, relative to the total weight of the composition.

### [Water]

The composition according to the present invention may further comprise water.

The amount of water in the composition according to the present invention may range from 10% to 90% by weight, preferably from 20% to 85% by weight, and more preferably from 30 to 80% by weight, relative to the total weight of the composition. The pH of the composition according to the present invention in this case may range from 2 to 7, preferably from 2 to 6 and more preferably from 2 to 4.

### [Buffering Agent]

It is preferable that the composition according to the present invention further comprise (d) at least one buffering agent. Two or more buffering agents may be used in combination. Thus, a single type of buffering agent or a combination of different types of buffering agents may be used.

The buffering agent can stabilize the pH of the composition according to the present invention.

As the buffering agent, an amino acid, amino acid derivative, or a combination of an organic acid or inorganic acid and a salt thereof, are preferable, and an amino acid and amino acid derivative are more preferable, and an amino acid is even more preferable.

The amino acids that may be used are of natural or synthetic origin, in L, D or racemic form, and comprise at least one acid function chosen from carboxylic acid, sulfonic acid, phosphonic acid, and phosphoric acid functions. The amino acids may be in their neutral or ionic form.

As amino acids, mention, in a nonlimiting manner, may be made of glycine, aspartic acid, glutamic acid, alanine, arginine, ornithine, citrulline, asparagine, carnitine, cysteine, glutamine, lysine, histidine, lysine, isoleucine, leucine, methionine, N-phenylalanine, proline, serine, taurine, threonine, tryptophan, tyrosine, and valine. Glycine is more preferable, because glycine may also function to reduce skin staining.

As examples of the organic acid, mention may be made of lactic acid, citric acid and malic acid.

As examples of the salt, mention may be made of alkaline metal salts such as sodium salt, potassium salt, alkaline earth metal salt such as calcium salt, and ammonium salt.

The amount of the (d) buffering agent(s) in the composition according to the present invention may range from 0.1 to 15% by weight, preferably from 0.5 to 10% by weight, and more preferably from 0.5 to 5% by weight, relative to the total weight of the composition.

### [Organic Solvent]

It is preferable that the composition according to the present invention further comprise (e) at least one organic solvent. Two or more organic solvents may be used in combination. Thus, a single type of organic solvent or a combination of different types of organic solvents may be used.

The organic solvent is preferably water miscible. As the organic solvent, there may be mentioned, for example, C₁-C₄ alkanols, such as ethanol and isopropanol; polyols and polyol ethers such as glycerol, 2-butoxyethanol, propylene glycol, monomethyl ether of propylene glycol, monoethyl ether and monomethyl ether of diethylene glycol; and aromatic alcohols such as benzyl alcohol and phenoxyethanol; analogous products; and mixtures thereof.

The amount of the (e) organic solvent(s) in the composition according to the present invention may range from 1 to 35% by weight, preferably from 5 to 25% by weight, and more preferably from 10 to 15% by weight, relative to the total weight of the composition.

### [Other Ingredients]

The pH of the composition according to the present invention may be adjusted to the desired value using acidifying or basifying agents commonly used in dyeing keratinous fibers or else using conventional buffer systems.

The composition according to the present invention is preferably acidic. Therefore, it is preferable that the pH of the composition be from 2 to 7, more preferably from 2 to 6, and even more preferably from 2 to 4.

Among the acidifying agents, mention may be made, by way of example, of mineral or organic acids such as hydrochloric acid, ortho-phosphoric acid, sulfuric acid, carboxylic acids such as acetic acid, tartaric acid, citric acid, and lactic acid, and sulfonic acids.

Among the basifying agents, mention may be made, by way of example, of ammonium hydroxide, alkali metal carbonates, alkanolamines such as mono-, di- and triethanolamines and also their derivatives, sodium or potassium hydroxide and compounds of the formula below:

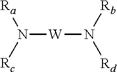

wherein
W denotes an alkylene such as propylene optionally substituted by a hydroxyl or a C₁-C₄ alkyl radical, and Rₐ, R_{b}, R_{c} and R_{d} independently denote a hydrogen atom, an alkyl radical or a C₁-C₄ hydroxyalkyl radical, which may be exemplified by 1,3-propanediamine and derivatives thereof. Sodium or potassium hydroxide is preferable, because this can also function to form in situ the (c) buffering agent.

The acidifying or basifying agent may be used in an amount ranging from 0.001 to 15% by weight, preferably from 0.01 to 10% by weight, and more preferably from 0.1 to 5% by weight, relative to the total weight of the composition.

The composition according to the present invention may comprise at least one thickening agent.

The thickening agent may be selected from organic and inorganic thickeners. The organic thickeners may be chosen from at least one of:
(i) associative thickeners;
(ii) crosslinked acrylic acid homopolymers;
(iii) crosslinked copolymers of (meth)acrylic acid and of (C₁-C₆)alkyl acrylate;
(iv) nonionic homopolymers and copolymers comprising at least one of ethylenically unsaturated ester monomers and ethylenically unsaturated amide monomers;
(v) ammonium acrylate homopolymers and copolymers of ammonium acrylate and of acrylamide;
(vi) polysaccharides such as cellulose and its derivatives; and
(vii) C₁₂-C₃₀ fatty alcohols.

The thickening agent is preferably selected from polysaccharides such as starch, xanthan gum, and hydroxyethylcellulose.

As used herein, the expression “associative thickener” means an amphiphilic thickener comprising both hydrophilic units and hydrophobic units, for example, comprising at least one C₈-C₃₀ fatty chain and at least one hydrophilic unit.

The viscosity of the composition according to the present invention is not particularly limited.

The viscosity can be measured at 25° C. with viscosimeters or rheometers preferably with coneplan geometry. Preferably, the viscosity of the composition according to the present invention can range, for example, from 1 to 2000 Pa·s, and preferably from 1 to 1000 Pa·s at 25° C. and 1 s⁻¹.

The thickening agent may be present in an amount ranging from 0.001 to 10% by weight, and preferably from 0.01 to 10% by weight, such as from 0.1 to 5% by weight, relative to the total weight of the composition.

The compositions according to the present invention may also contain various adjuvants conventionally used in compositions for dyeing hair, such as anionic, non-ionic, cationic, amphoteric or zwitterionic polymers, or mixtures thereof, antioxidants, penetrating agents, sequestering agents, fragrances, dispersing agents, conditioning agents, film-forming agents, ceramides, preservatives and opacifying agents.

The form of the composition according to the present invention is not particularly limited, as long as it is water-based, and may take various forms such as an emulsion, an aqueous gel, an aqueous solution, or the like.

The composition according to the present invention is a composition for dyeing keratin fibers, and is preferably a cosmetic composition for dyeing keratin fibers. The “keratin fibers” here mean fibers which include at least one keratin substance. It is preferable that at least a part of the surface of the keratin fibers be formed by keratin fibers. Examples of keratin fibers include hair, eyebrows, eyelashes, and the like. It is preferable that the composition according to the present invention be used for dyeing hair.

### [Preparation]

The composition according to the present invention can be prepared by mixing (a) at least one direct dye, and (b) at least one oxygen-containing polycyclic compound, as essential ingredients, as well as optional ingredient(s) as explained above.

The method and means to mix the above essential and optional ingredients are not limited. Any conventional method and means can be used to mix the above essential and optional ingredients to prepare the composition according to the present invention.

The composition according to the present invention is preferably a so-called one-part composition or a ready-to-use composition. For the purposes of the present invention, the expression “ready-to-use composition” is defined herein as a composition to be applied immediately to keratin fibers such as hair.

As compared to a so-called two-part composition, a so-called one-part composition does not need to mix ingredients in the composition prior to use. Therefore, it is easy for a consumer to use the composition according to the present invention for dyeing keratin fibers.

Furthermore, stable coloring of keratin fibers is possible for the composition according to the present invention, because it is not possible to fail to mix ingredients in a precise mixing ratio which is required for two-part compositions for dyeing keratin fibers.

### [Process]

The present invention also relates to a process for dyeing keratin fibers, comprising the step of applying the composition according to the present invention to the keratin fibers.

The step of applying the composition according to the present invention to the keratin fibers can be performed by a conventional applicator such as a brush, or even by the hands.

The keratin fibers to which the composition according to the present invention has been applied can be left for an appropriate time which is required to treat the keratin fibers. The time length for the treatment is not limited, but it may be from 1 minute to 1 hour, preferably 1 minute to 30 minutes, and more preferably 1 minute to 15 minutes. For example, the time for dyeing the keratin fibers may be from 1 to 20 minutes, preferably 5 to 15 minutes.

The keratin fibers may be treated at room temperature. Alternatively, the keratin fibers can be heated at 25° C. to 65° C., preferably 30° C. to 60° C., more preferably 35° C. to 55° C., and even more preferably 40° C. to 50° C., during the step of applying the composition according to the present invention to the keratin fibers, and/or the step of leaving the keratin fibers to which the composition according to the present invention has been applied.

The keratin fibers may be rinsed after the step of applying the composition according to the keratin fibers onto the keratin fibers and/or after the step of leaving the keratin fibers to which the composition according to the present invention has been applied.

The present invention may also relate to the use of the composition according to the present invention for dyeing keratin fibers such as hair.

### Examples

The present invention will be described in a more detailed manner by way of examples. However, these examples should not be construed as limiting the scope of the present invention.

### Examples 1-2 and Comparative Examples 1-2

### [Preparation]

Each of the cosmetic compositions for dyeing hair according to Examples 1-2 (Ex. 1 and Ex. 2) and Comparative Examples 1-2 (Comp. Ex. 1 and Comp. Ex. 2) was prepared by mixing the ingredients shown in Table 1 at room temperature, and were poured into transparent vessels with the same volume. The numerical values for the amounts of the ingredients are all based on “% by weight” as active raw materials.

### [Evaluation of Color Difference]

Each of the compositions according to Examples 1-2 and Comparative Examples 1-2 was evenly applied onto 1 g of a tress of 100% white natural human hair. The tress was then left for 15 minutes at 40° C., followed by washing with water, shampooing, rinsing once and drying the tress. The difference in color of the tress before and after the above dyeing process was evaluated by using Minolta CM-580. ΔE* (between the color of the undyed original tress and the color of the dyed tress based on CIE1976) was calculated. The larger ΔE* is, the better the dyeing is. The results are shown in Table 1.

### [Evaluation of Skin Staining]

The skin staining of the composition was subjected to the sensory evaluation by 10 experts. Each of the compositions according to Examples 1-2 and Comparative Examples 1-2 was applied onto the surface of a forearm of 10 human experts. The applied surface was left for 15 minutes at room temperature, followed by thoroughly washing out the composition with water, and drying the surface. Evaluation was carried out in accordance with the following criteria.
A: At least 80% of 10 experts recognized skin staining to be absolutely unnoticeable.
B: At least 50% but less than 80% of 10 experts recognized skin staining to be absolutely unnoticeable.
C: At least 20% but less than 50% of 10 experts recognized skin staining to be absolutely unnoticeable.
D: Less than 20% of 10 experts recognized skin staining to be absolutely unnoticeable.

The results are shown in Table 1.

TABLE 1

| | | | Comp. | Comp. |
|---|---|---|---|---|
| | Ex. 1 | Ex. 2 | Ex. 1 | Ex. 2 |
| Hydroxyethylcellulose | 1.5 | 1.5 | 1.5 | 1.5 |
| CI 60730 (Acid Violet 43) | 0.07 | 0.07 | 0.07 | 0.07 |
| Acid Black 1 | 0.07 | 0.07 | 0.07 | 0.07 |
| CI 15510 (Acid Orange 7) | 0.25 | 0.25 | 0.25 | 0.25 |
| Lactic acid | 2.0 | 2.0 | 2.0 | 2.0 |
| Glycine | 1 | 1 | 1 | 1 |
| Piperonyl Alcohol | 3 | 3 | — | — |
| Dimethyl Sulfone | — | 6 | — | 6 |
| Benzyl Alcohol | — | — | 3 | 3 |
| Alcohol Denat. | 20 | 10 | 20 | 10 |
| Water | qs 100 | qs 100 | qs 100 | qs 100 |
| ΔE* | 50.5 | 49.8 | 50.1 | 49.5 |
| Skin Staining | B | A | D | C |

It can be recognized from the comparisons of Example 1 and Comparative Example 1 and of Example 2 and Comparative Example 2, shown in Table 1 that, when a cosmetic composition for dyeing hair containing a direct dye further contains piperonyl alcohol, the cosmetic composition can prevent or reduce skin staining by the direct dye on the skin such as the scalp, while maintaining good cosmetic effects such as good coloring properties.

It can be recognized from the comparison of Example 1 and Example 2 shown in Table 1 that, when a cosmetic composition for dyeing hair containing a direct dye further contains a sulfone compound in addition to piperonyl alcohol, the cosmetic composition can furthermore prevent or reduce skin staining by the direct dye on the skin such as the scalp, while maintaining good cosmetic effects such as good coloring properties.

1-15. (canceled)

16. A composition for dyeing keratin fibers, comprising:
(a) at least one direct dye; and
(b) at least one oxygen-containing polycyclic compound comprising at least one oxygen-containing polycyclic ring.

17. The composition according to claim 1, wherein the at least one direct dye is chosen from acidic direct dyes, basic direct dyes, neutral direct dyes, or mixtures thereof.

18. The composition according to claim 1, wherein the at least one direct dye is chosen from:
diaryl anionic azo dyes according to formula (II) or (II′) below: 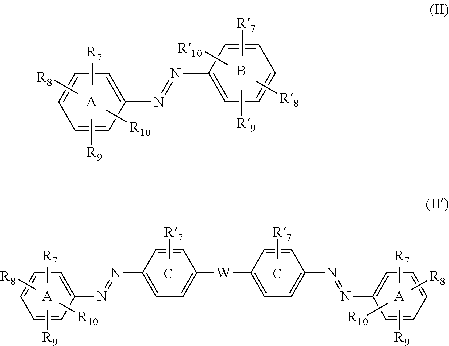
wherein
R₇, R₈, R₉, R₁₀, R′₇, R′₈, R′₉, and R′₁₀, which may be identical or different, are chosen from a hydrogen atom or a group chosen from:
alkyl;
alkoxy, alkylthio;
hydroxyl, mercapto;
nitro;
R^{º}—C(X)—X′—, R^{º}—X′—C(X)—, R^{º}—X′—C(X)—X″—, wherein R^{º} is chosen from a hydrogen atom or an alkyl or aryl group; X, X′ and X″, which may be identical or different, are chosen from an oxygen or sulfur atom, or NR, wherein R is chosen from a hydrogen atom or an alkyl group;
(O)₂S(O⁻)—, X⁺, wherein X⁺ is chosen from an organic or mineral cationic counter ion;
(O)CO⁻—, X⁺;
(O)P(O₂⁻)—, 2X⁺;
R″—S(O)₂—, wherein R″ is chosen from a hydrogen atom or an alkyl, aryl, (di)(alkyl)amino, or aryl(alkyl)amino group;
R′″—S(O)₂—X′—, wherein R′″ is chosen from an alkyl or optionally substituted aryl group;
(di)(alkyl)amino;
aryl(alkyl)amino optionally substituted with at least one group chosen from nitro, nitroso, (O)₂S(O⁻)—, X⁺, or alkoxy with X⁺;
optionally substituted heteroaryl;
cycloalkyl;
Ar—N═N—, wherein Ar is chosen from an optionally substituted aryl group;
or alternatively two contiguous groups R₇ with R₈ or R₈ with R₉ or R₉ with R₁₀ together form a fused benzo group A′; and R′₇ with R′₈ or R′₈ with R′₉ or R′₉ with R′₁₀ together form a fused benzo group B′; with A′ and B′ optionally substituted with at least one group chosen from nitro, nitroso, (O)₂S(O⁻)—, X⁺; hydroxyl, mercapto, (di)(alkyl)amino, R^{º}—C(X)—X′—, R^{º}—X′—C(X)—, R^{º}—X′—C(X)—X″—, Ar—N═N—, or optionally substituted aryl(alkyl)amino; and
W is chosen from a sigma bond σ, an oxygen or sulfur atom, or a divalent radical —NR— or methylene —C(Rₐ)(R_{b})—, wherein Rₐ and R_{b}, which may be identical or different, are chosen from a hydrogen atom or an aryl group, or alternatively Rₐ and R_{b} form, together with the carbon atom that bears them, a spiro cycloalkyl;
wherein formulae (II) and (II′) comprise at least one sulfonate (O)₂S(O⁻)—, X⁺, phosphonate (O)P(O₂⁻) 2X⁺, or carboxylate (O)C(O⁻)—, X⁺ radical on one of the rings A, A′, B, B′, or C;
anthraquinone dyes according to formulae (III) and (III′): 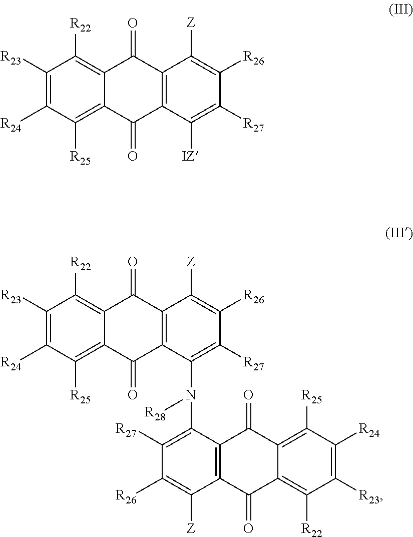
wherein:
R₂₂, R₂₃, R₂₄, R₂₅, R₂₆, and R₂₇, which may be identical or different, are chosen from a hydrogen atom, a halogen atom, or a group chosen from:
alkyl;
hydroxyl, mercapto;
alkoxy, alkylthio;
aryloxy or arylthio optionally substituted, preferably substituted with at least one group chosen from alkyl or (O)₂S(O⁻)—, X⁺, wherein X⁺ is chosen from an organic or mineral cationic counter ion;
aryl(alkyl)amino optionally substituted with at least one group chosen from alkyl or (O)₂S(O⁻)—, X⁺;
(di)(alkyl)amino;
(di)(hydroxyalkyl)amino; or
(O)₂S(O⁻)—, X⁺;
Z′ is chosen from a hydrogen atom or a group NR₂₈R₂₉ wherein R₂₈ and R₂₉, which may be identical or different, are chosen from a hydrogen atom or a group chosen from:
alkyl;
polyhydroxyalkyl;
aryl optionally substituted with at least one group chosen from alkyl, (O)₂S(O⁻)—, X⁺, R^{º}—C(X)—X′—, R^{º}—X′—C(X)—, or R^{º}—X′—C(X)—X″—; or
cycloakyl;
Z is chosen from hydroxyl or NR′₂₈R′₂₉, wherein R′₂₈ and R′₂₉, which may be identical or different, are chosen from a hydrogen atom or a group chosen from:
alkyl;
polyhydroxyalkyl; or
aryl optionally substituted with at least one group, chosen from alkyl, (O)₂S(O⁻)—, X⁺, R^{º}—C(X)—X′—, R^{º}—X′—C(X)—, or R^{º}—X′—C(X)—X″—; and
cycloakyl;
wherein formulae (III) and (III′) comprise at least one sulfonate group (O)₂S(O⁻)—, X⁺;
or
quinoline-based dyes according to formula (IV): 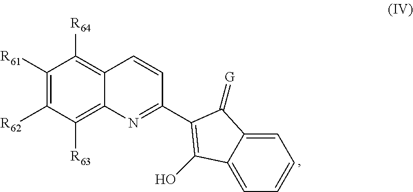
wherein:
R₆₁ is chosen from a hydrogen atom, a halogen atom, or an alkyl group;
R₆₂, R₆₃, and R₆₄, which may be identical or different, are chosen from a hydrogen atom or a group (O)₂S(O⁻)—, X⁺, wherein X⁺ is chosen from an organic or mineral cationic counter ion;
or alternatively R₆₁ with R₆₂, or R₆₁ with R₆₄, together form a benzo group optionally substituted with at least one (O)₂S(O⁻)—, X⁺ group; and
G is chosen from an oxygen atom, a sulfur atom, or a NRₑ group, wherein Rₑ is chosen from a hydrogen atom or an alkyl group;
wherein formula (IV) comprises at least one sulfonate group (O)₂S(O⁻)—, X⁺.

19. The composition according to claim 16, wherein the at least one direct dye is present in an amount ranging from about 0.001% to about 5% by weight, relative to the total weight of the composition.

20. The composition according to claim 16, wherein the at least one direct dye is present in an amount ranging from about 0.05% to about 2% by weight, relative to the total weight of the composition.

21. The composition according to claim 16, wherein the at least one oxygen-containing polycyclic ring is chosen from a phthalan ring, coumaran ring, isobenzofuran ring, coumaron ring, chroman ring, chromen ring, isochromen ring, or piperonyl ring.

22. The composition according to claim 16, wherein the at least one oxygen-containing polycyclic ring is chosen from a hydroxyl group; an optionally substituted, linear or branched C₁₋₁₈ alkyl group; an optionally substituted, linear or branched C₁₋₁₈ alkoxy group; an optionally substituted C₃₋₁₈ alicyclic alkyl group; an optionally substituted, linear or branched C₂₋₁₈ alkenyl group; an optionally substituted, linear or branched C₂₋₁₈ alkenyloxy group; an optionally substituted, linear or branched C₂₋₁₈ alkynyl group; an optionally substituted, linear or branched C₂₋₁₈ alkynyloxy group; an optionally substituted C₆₋₁₈ aryl group; an optionally substituted C₇₋₁₈ aralkyl group; or an optionally substituted, linear or branched C₂₋₁₈ acyl group.

23. The composition according to claim 16, wherein the at least one oxygen-containing polycyclic compound is chosen from compounds according to chemical formula (1) below: 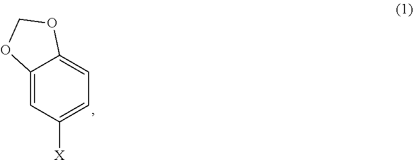
wherein:
X is chosen from a hydroxyl, —(CH₂)ₙ—R, or —(CH₂)ₙ—O—R, wherein n is an integer ranging from 1 to 10, and R is chosen from a hydrogen, an optionally substituted, linear or branched C₁₋₁₈ alkyl group, an optionally substituted C₃₋₁₈ alicyclic alkyl group, an optionally substituted, linear or branched C₂₋₁₈ alkenyl group, an optionally substituted, linear or branched C₂₋₁₈ alkynyl group, an optionally substituted, C₆₋₁₈ aryl group, an substituted C₇₋₁₈ aralkyl group, or an optionally substituted, linear or branched C₂₋₁₈ acyl group.

24. The composition according to claim 16, wherein the at least one oxygen-containing polycyclic compound is chosen from piperonyl alcohol, sesamol, heliotropyl acetone, or heliotropyl acetate.

25. The composition according to claim 16, wherein the at least one oxygen-containing polycyclic compound is present in an amount ranging from about 0.001% to about 15% by weight, relative to the total weight of the composition.

26. The compound according to claim 16, wherein the at least one oxygen-containing polycyclic compound is present in an amount ranging from about 0.05% to about 5% by weight, relative to the total weight of the composition.

27. The composition according to claim 16, further comprising at least one sulfone compound according to formula (I): 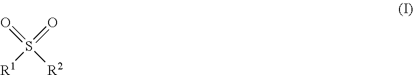
wherein:
each of R¹ and R², independently of each other, are chosen from a monovalent C₁₋₃₀ aliphatic group or a monovalent C₆₋₃₀ aromatic group, which may optionally be substituted with at least one substituent; or
R¹ and R², together with the sulfur atom which they are binding, form a 3-10 membered ring which may optionally be substituted with at least one substituent.

28. The composition according to claim 27,
wherein the monovalent C₁₋₃₀ aliphatic group is chosen from:
a saturated monovalent C₁₋₃₀ aliphatic hydrocarbon group, or
an unsaturated monovalent C₂₋₃₀ aliphatic hydrocarbon group;
wherein the monovalent C₆₋₃₀ aromatic group is a monovalent C₆₋₃₀ aromatic hydrocarbon group.

29. The composition according to claim 27, wherein the at least one sulfone compound is present in an amount ranging from about 0.5% to about 30% by weight, relative to the total weight of the composition.

30. The composition according to claim 27, wherein the at least one sulfone compound is present in an amount ranging from about 2% to about 10% by weight, relative to the total weight of the composition.

31. The composition according to claim 16, further comprising water.

32. A method for dyeing keratin fibers, comprising applying a composition to the keratin fibers, the composition comprising:
(a) at least one direct dye; and
(b) at least one oxygen-containing polycyclic compound comprising at least one oxygen-containing polycyclic ring.
